SEQUENCE LISTING (10 sequences)

SEQ ID NO: 1 - protein, 937 aa, Homo sapiens
CCACCTACACGAGGGCGCCCCCATCTTATGGTGGAAGCAGTCGCTATGATGATTACAGCAGCTCACGTGACGGATATGGTGGAAGTCGAGACAGTTACTCAAGCAGTCGAAGTGATCTCTACTCAAGTGGTCGTGATCAGGTTGGCAGACAAGAAAGAGGGCTTCCCCCTTCTATGGAAAGGGGGTACCCTCCTCCACGTGATTCCTACAGCAGTTCAAGCCGTGGAACACCAAGAGGTGGTGGCCGTGGAGGAAGCCGATCTGATAGAGGGGGAGGCAGAAGCAGATACTAGAAACAAACAAAACTTTGTACCAAAATCCCAGTTCAAAGAAACAAAAAGTGGAAACTATTCTATCATAACTACCCAAGAACTACTAAAAGGAAAAATTGTGTTACCTTTTTTAAAATTCCCTGTTAAGCTCCCCTCCATAATTTTTATGTTCTTGTGAGGAAAAAAAGTAAAACATGTTTAATTTTATTTGACTTTTGCATTGCTTTTCAACAAGCAAATGTTAAATGTGTTAAGACTTATACTAGTGTTGTAACTTTCCAAGTAAAAGTATCCCTAAAGGCCACTTCCTATCTGATTTTTCCCAGTAAATGAGGCAGGCAATTCTAAGATCTTCCACAAAACATCTAGCCATCTAAAATGGAGAGATGAATCATTCTACCTACACAAACAAGCTAGCTATTAGAGGGTGGTTGGGATATGCTACTCATAAGATTTCAGGGTGTCTTCCAACTGAAATCTCAATGTTCTTAGTATGAAAAACCTGAAATCGCATGCCTATTCACCCAGTAAACCCAAAAAAGCAAATGGATAATGCTGGCCATTCTGCCTTTCTGACATTTCCTTGGGAATCTGCAAGAACCTCCCCTTTCCCCTCCCCCAATAAGACCATTTAAGTGTGTGCTAAACAACTAAGAATACTAAAT

SEQ ID NO: 2 - protein, 342 aa, Homo sapiens
GAGATGGTGCCCTTGATTAGAAGTGTCTGGAGGGGGATAAATGGAGGGGATAAGATTCAGTTGGTTTTGGAAAATGTTAAAGTCTTAAAATAATGCGTCCATCTGAAGAATTTTTTCTAAAACCAGAGTTTATAAAAATATCACTGATACAGCCTGCCCCCTCATTTCCCTGCCACAGGAGATGTCTTGGACTAGAGACACTTGTTTAATAATAGCTTGTCTCTGATATTCCCAGTAGCTTCCCTCTGTGTGAGGAAAGGATAGAAATGTTCAGGACATCATCATACAGGCTCCTCATCTACAAAGTTCCAGTAGCAGTGACGCCTACACGGAAGACTTGGA

SEQ ID NO: 3 - protein, 47 aa, Homo sapiens
GTGTGACTGAAGAAATATCAAATGTTTCCTAGTAAGACAGCAACTCA

SEQ ID NO: 4 - protein, 46 aa, Homo sapiens
ACTCTAGGATGGAAGAAGGTGTCTGACCGTAAATTACACCTGCAGT

SEQ ID NO: 5 - protein, 160 aa, Homo sapiens
AACCAGCAGACTAATGGGGATGAGGTTCTGGTACAAGATGATGAACACCAGTATGTCAGACAATGACTTGGGAGCTGGAATCAAGGACATGACCAAGAGCAGCAAGAACAAAAGGGAGACTGACACATTGATCACTTTCTCAACCTTTGATCTCTTGAGA

SEQ ID NO: 6 - protein, 120 aa, Homo sapiens
ATGGTTCACATTTGAGTAAAGACAGGGGAGTTTGTTTTCAGAATGACATACTAGTCTGCAGGATGAATTTCATAACTGACATTGCACCTTGGACTGCAACTAGGACTTTCACTGGAATCA

SEQ ID NO: 7 - protein, 1737 aa, Homo sapiens
GAAAGAGTTTTGAAGAAAACTGGGCATAGGCTCAGCAAAACCAAACAGAAGAGGAACAGAAAAAGAAACAAAAAGCAGAACAGTCAGAATAGAATCATGGAGGAAAACTCATTAGAATTCTTAAGTGATCTTACACCGGGAGATCAGGACCCATCTCAGAGTGAAGAGGAAGACATTGAAAAGACCAGAAGAGAATCAGAATATCCCTTCATTGATGGTCTACAAAATGAAGTCGGAGATTTTGTGACTGGATATAAAGAAAAAAGATGGAAAAATAAAGATCCTAAAGACAGTTTCCAAAACGTTATGTCTATAGTTGAATTAGACAACACACCAAAGAATTACCTCTCTAAGGAAGGTGATAACTTGTTTGTAAGTTTGTTACTGAGGCCAAATGAAATCTCCGTTACTTGTCCAATACTGACTCAAAACCTTTCCTGTGTAACAACTGATGACTGCTCTGGCATGAAGGTAGAAAAGCATATTAGAAATAGGCATACCATAGCATTAGACACCCAGGACCTTTCTGCGGAAACTTCATGCTTATTTATGAAGAAGAGAGAAATAGTAGATAAAAATCTCTCACATGAACCCATTCTGTGCCATCAACATGGAATCAGAATGTCAGATAAAGTTTTAAGAGAGGAACAAGTGTATACAACTAAAATCAATCACTGGGCTTTTTTCACAACCAATTTATCTGATGAAGATTTACAGCTGGGCTCTGACAGACAGCCCTATTTTGGTAGCTGGCCTGCAGGACCTCATAAGTTTATATGTGAACAGAGACCAAAGAAAGATAGAGCATGTAAGTTGGCTGGTCCTGACAGCAGGGGGCAATGGATTCAAATGATCTTCACTTCGGTGGCAGCATCAGAACCAGGAAACAATCCAGAAATATTGACAGACAAACTACTGATAGGAAATGAAGATTTTTCACCTCCACCTGAAACTATGGATTCATTCATAGAAACAAACCTCTTCAGAAGCTGCTTACCTCAACCGGATATACCAAAGAATGCCTTAGAATCAACAAAAAATAAGAAAAGGAGGAAGAAAAGGATTTTCAATTTGGTACCAAATTTTGACTTATTAGGACAGAGTCGTATCGGTGTAAAAGAAAGGGAGAAATGTGACCTGTTAACAAAAAACCATGGACTAAAAATTACTTTGGGAGAAGAAAAAGATAGAATTTCAGAAAGGAACAGTGAAGAGGAGAATAAACAAAAACTTATGACCTTTGATCATCATCCATTGTGGTTTTACCTTGATATTATCAAAGCTACCCCTTTAAATATTGATGGACAGCGTTATTCTCATTGCCTGTCATTTAACAGACTAAGGTGCTCTGCATCTTTATACAAAAATTATATTCCTTCTTTTGTGCTACATAATTTATCTAGTATTTGGAAGCCATCTTTTACAAACAAGAAACTGTTTTTGACTTTCGAATCTCAGACAAGAGTAGGTAATAAACTAAATGATGCAGGGTTTATTTCTCCAGAAATTTTACATAGTCATCCTGATACTTCGTGCTCTTTGGGAGTCACTTCTGATTTTCACTTTTTAAATGAAAGGTTTGATAGAAAGCTGAAAAGATGGGAAGAACCTAAGGAATTACCAGCTGAGGACAGCCAAGACTTAACAAGCACTGACTACCGTTCCCTTGAGCTACCATTATCACAAGGGTTTGCCTTTCAATTAGTAAAGCTTTTTGGATCTCCAGGCGTTCCAATG

SEQ ID NO: 8 - protein, 84 aa, Homo sapiens
ATCCTTGTTGCCTGATGACTATGTGGTTCCCCTTGACTGGAAGACACTAAAGATGATCTACTTGCAATGGAAGATGTCAGTGGA

SEQ ID NO: 9 - protein, 43 aa, Homo sapiens
AAAGACAGAAGAAGATTGGTTGAAAAATGAAAATTCCTTGAAC

SEQ ID NO: 10 - protein, 554 aa, Homo sapiens
TGAGTTCTGCTGTCTTCATGGTACTGCTGAAGATCATGATCACGGAGAAAAGTCAGAGTGCTCAGTGCCAACCCAAGGGATTCTTTCCAGAGACGTACCCGTTGGATACCAAAATTAGTTTGGATAATCTGTTCAACCATTCTTGATAAGTTATCTGAATAATAAAAAAACTCAACAGAGGAGGTAACAATTTGAACATTTTATTGTCTAATTTGAAGATGTATGCCATACTTTGTTTGATAGAAGAAAGTAAGGCACAGAAAACTTGAGTACCTTATTTTTAAAACTGCATTAGGATTAAAAGGTTAGCCCCTATATCCAAGTATTGGTCTGAGATCCCATTTCTAGAATTCTGAAATCCAAAAAGCTCTGAAAATCAATAGTTATTTTTCCAAATGTATTCATTGTGGTAAAATACACCAATATAAAATTTACCATCTTAACCACTTTTAAGTGTGTTATAAATACATTCATGCTACCATCACTAGCATCCATCTCTGGAACTCTTTTCATCTTGCAAAACTGCAATTCTATACCCATTAAACAATGACCCA

(19) United States
(12) Patent Application Publication
(10) Pub. No.: US 2017/0049815 A1
(43) Pub. Date: Feb. 23, 2017

(54) Selective Chemotherapy Treatments and Diagnostic Methods Related Thereto

(71) Applicant: Emory University, Atlanta, GA (US)

(72) Inventor: Taofeek Kunle Owonikoko, Atlanta, GA (US)

(21) Appl. No.: 15/308,493

(22) PCT Filed: May 1, 2015
(86) PCT No.: PCT/US2015/028784

§ 371 Date: Nov. 2, 2016

Related U.S. Application Data
(60) Provisional application No. 61/987,885, filed on May 2, 2014.

(51) Int. Cl. A61K 33/24; C12Q 1/68; A61K 31/7048; A61K 31/4184

(52) U.S. Cl. CPC A61K 33/24; A61K 31/4184; C12Q 1/6886; A61K 31/7048; C12Q 2600/106; C12Q 2600/158

(57) ABSTRACT

This disclosure relates to methods of identifying subjects that have an increased likelihood of responding to a combination of a poly (ADP) ribose polymerase enzyme inhibitor and a platinum based reagent and optionally other anticancer agents in the course of chemotherapy. In certain embodiments, the disclosure relates to methods of treating cancer comprising administering an effective amount of a poly (ADP) ribose polymerase enzyme inhibitor and a platinum based reagent to the subject in need thereof, wherein the subject is in need thereof because measuring a quantity of RNA isolated from a cancer cell from the subject indicates an increased quantity of the RNA compared to a normal sample, wherein the RNA is associated with one or more of the following genes/pseudogenes GLS, UBEC2, HACL1, MSI2, and LOC100129585.

### CROSS REFERENCE TO RELATED APPLICATIONS

This application claims priority to U.S. Provisional Application No. 61/987,885 filed May 2, 2014, hereby incorporated by reference in its entirety.

### STATEMENT REGARDING FEDERALLY FUNDED RESEARCH

This invention was made with government support under Grants 1K23CA164015 and P01CA116676 awarded by the National Institutes of Health. The Government has certain rights in the invention.

### BACKGROUND

Small cell lung cancer (SCLC) is a lethal disease with limited treatment options. The standard frontline therapy is the combination of platinum and topoisomerase inhibitor. While efforts to identify promising targeted biologic agents for the treatment of this disease continue, cytotoxic chemotherapy remains the mainstay of treatment. Thus, there is a need to identify improvement therapeutic methods.

Poly (ADP) ribose polymerase (PARP) is a family of enzymes that catalyze the addition of ADP-ribose to a variety of cellular constituents. PARP is involved in DNA damage repair, primarily through base excision repair (BER) mechanism, important cellular machinery for repairing single strand breaks typically induced by cytotoxic therapeutic agents for small cell lung cancer (SCLC). Veliparib (ABT-888) is a small molecule inhibitor of PARP-1 and PARP-2. Donawho et al. report veliparib potentiates DNA-damaging agents in preclinical tumor models. Clin. Cancer Res, 2007, 13:2728-2737.

Byers et al. report proteomic profiling identifies dysregulated pathways in small cell lung cancer and therapeutic targets including PARP1. Cancer Dis, 2012, 2:798-811.

References cited herein are not an admission of prior art.

### SUMMARY

This disclosure relates to methods of identifying subjects that have an increased likelihood of responding to a combination of a poly (ADP) ribose polymerase enzyme inhibitor and a platinum based reagent and optionally other anticancer agents in the course of chemotherapy. In certain embodiments, the disclosure relates to methods of treating cancer comprising administering an effective amount of a poly (ADP) ribose polymerase enzyme inhibitor and a platinum based reagent to the subject in need thereof, wherein the subject is in need thereof because measuring a quantity of RNA isolated from a cancer cell from the subject indicates an increased quantity of the RNA compared to a normal sample, wherein the RNA is associated with one or more of the following genes/pseudogenes GLS, UBEC2, HACL1, MSI2, and LOC100129585.

In certain embodiments, the poly(ADP) ribose polymerase enzyme inhibitor is veliparib. In certain embodiments, the platinum based reagent is cisplatin. In certain embodiments, cancer is lung cancer.

In certain embodiments, the disclosure relates to methods of diagnosing a subject as a candidate for treatment with a poly (ADP) ribose polymerase enzyme inhibitor and a platinum based reagent comprising measuring a quantity of RNA isolated from a cancer cell from the subject wherein the measurement indicates an increased quantity of the RNA compared to a normal sample, wherein the RNA is associated with one or more of the following genes/pseudogenes: GLS, UBEC2, HACL1, MSI2, and LOC100129585 and correlating the increased quantity to a diagnoses that the subject is candidate for treatment with a poly (ADP) ribose polymerase enzyme inhibitor and a platinum based reagent.

In certain embodiments, the disclosure relates to methods of diagnosing a subject as not a candidate for treatment with a poly (ADP) ribose polymerase enzyme inhibitor and a platinum based reagent comprising measuring a quantity of RNA isolated from a cancer cell from the subject wherein the measurement indicates an increased quantity of the RNA compared to a normal sample, wherein the RNA is associated with one or more of the following genes/pseudogenes CENPE, CRYGS, FAM83D, FLJ44342, GNA12, LOC88523, LRDD, N4BP2L2, SLC35A3, SPC25 and correlating the increased quantity to a diagnoses that the subject is not candidate for treatment with a poly (ADP) ribose polymerase enzyme inhibitor and a platinum based reagent.

### BRIEF DESCRIPTION OF THE FIGURES

FIG. 1A shows data indicating veliparib showed limited single-agent activity across a wide concentration range in a panel of SCLC cell lines.

FIG. 1B shows the log of mean±SEM of IC₅₀ concentrations for cisplatin, carboplatin, and etoposide alone and in combination with 5 and 50 μmol/L concentrations of veliparib. Each bar represents log of the mean value obtained from 3 to 4 independent experiments. Bottom Right: Potentiation of cytotoxicity induced by gamma radiation in the presence of veliparib (5 μmol/L) in 2 representative cell lines (H146 and DMS153).

FIG. 2A shows data on H146 tumor-bearing animals that were treated as indicated with vehicle, veliparib alone, cisplatin alone, and the combination of veliparib and cisplatin. Subcutaneous tumor volumes were measured at least twice weekly. The combination of veliparib with cisplatin induced greater tumor growth inhibition than cisplatin alone.

FIG. 2 B shows data indicating animals treated with the combination of veliparib and cisplatin had the smallest tumor burden as indicated by the weights of tumor tissue harvested from euthanized mice at the end of the experiments.

FIG. 2C shows data on H128 xenografts that were raised in nu/nu mice. Tumor-bearing animals were treated as indicated with vehicle, veliparib alone, cisplatin alone, and the combination of veliparib and cisplatin. Subcutaneous tumor volumes were measured at least twice weekly. The combination of veliparib with cisplatin did not induce significantly greater tumor growth inhibition than cisplatin alone, similar to in vitro observations in the H128 cell line.

FIG. 2D shows data indicating the addition of veliparib to cisplatin did not result in reduced tumor burden as indicated by the comparable weights of tumor tissue harvested from animals treated with cisplatin alone or with the combination of cisplatin and veliparib at the end of the experiments.

FIG. 3A shows data on tumor growth curves indicating greater growth inhibition by doublet and triplet regimen during active treatment period (Weeks 1-4) wherein CDDP is cisplatin, VP16 is etoposide, and ABT is veliparib.

FIG. 3B shows data indicating greater delay in tumor regrowth in animals treated with the triplet when observed off treatment (Weeks 4-9).

FIG. 3C showing different tumor regrowth kinetic between doublet (cisplatin and etoposide) and triplet treatment (veliparib [25 mg/kg], cisplatin [2.5 mg/kg i.p., weekly] and etoposide [20 mg/kg i.p., weekly]); P<0.021.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

In this specification and in the claims that follow, reference will be made to a number of terms that shall be defined to have the following meanings unless a contrary intention is apparent.

Prior to describing the various embodiments, the following definitions are provided and should be used unless otherwise indicated.

The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

The term “antisense” refers to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex which is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA and that blocks the expression of a target gene by interfering with the processing, transport and/or translation of its primary transcript or mRNA. The complementarity of an antisense RNA may be with any part of the specific gene transcript, i.e., at the 5′ non-coding sequence, 3′ non-coding sequence, introns, or the coding sequence. In addition, as used herein, antisense RNA may contain regions of ribozyme sequences that increase the efficacy of antisense RNA to block gene expression.

The term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture. This process for amplifying the target sequence consists of introducing a large excess of two oligonucleotide primers to a mixture of nucleic acids containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). Any oligonucleotide or polynucleotide sequence can be amplified with the appropriate set of primer molecules. In particular, the amplified segments created by the PCR process itself are, themselves, efficient templates for subsequent PCR amplifications.

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

The term “reverse-transcriptase” or “RT-PCR” refers to a type of PCR where the starting material is RNA. The starting RNA is enzymatically converted to complementary DNA or “cDNA” using a reverse transcriptase enzyme. The cDNA is then used as a “template” for a “PCR” reaction The term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and into protein, through “translation” of RNA.

The term “immobilized” when used in reference to nucleic acid refers to a spatial restriction of the nucleic acid on a surface, which restriction prevents the nucleic acid from entering the solution in which the surface is located and becoming free in the solution; it involves stable complex formation, where the complex comprises the nucleic acid and formation of the complex is mediated at least in part by electrostatic interactions

“Subject” means any animal, but is preferably a mammal, such as, for example, a human, monkey, mouse, or rabbit.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

### Gene Panel as Biomarkers Indicating Poly (ADP) Ribose Polymerase Enzyme Inhibitor, Veliparib, Potentiates Chemotherapy and Radiation

Experiments herein indicate that the strategy of targeting PARP enzyme as a potential therapy of SCLC. Initial attempts at clinical translation of PARP inhibition relied on a strategy of synthetic lethality targeting genetically vulnerable tumors such as BRCA1- and BRCA2-deficient breast and ovarian cancers. The limitations of such an approach have become apparent due to limited efficacy of single-agent PARP inhibitor therapy. The proficient DNA damage repair capability of cancer cell lines when exposed to ionizing radiation and chemotherapeutic agents has been shown to correlate with treatment resistance. Given the central role of PARP enzyme in DNA damage recognition and subsequent repair by BER and its potential role in homologous recombination repair (HRR), the use of a PARP inhibitor to impede the ability of cancer cells to repair DNA damage induced by cytotoxic agents is a rational approach under intensive preclinical and clinical evaluation. Whether a pharmacologic PARP inhibitor, veliparib, in combination with DNA damaging agents could potentiate therapeutic efficacy in preclinical models of SCLC was explored.

A significant reduction in the level of PARylated proteins in cells treated with veliparib was observed at a concentration of 5 μmol/L but optimal therapeutic potentiation when combined with DNA damaging agents indicated a need for a much higher concentration of the compound. The intratumoral veliparib concentration of 2 μmol/L was determined to be sufficient for in vivo potentiation of the antitumor effect of cisplatin. Increased intratumoral platinum concentration was observed in the presence of veliparib.

The expression profile of a 5-gene panel identified may be used to predict both platinum sensitivity and PARP inhibitor efficacy in SCLC and potentially other tumor types.

In certain embodiments, the disclosure relates to methods of treating cancer comprising administering an effective amount of a poly (ADP) ribose polymerase enzyme inhibitor and a platinum based reagent to the subject in need thereof, wherein the subject is in need thereof because measuring a quantity of RNA isolated from a cancer cell from the subject indicates an increased quantity of the RNA compared to a normal sample, wherein the RNA is associated with one or more of the following genes GLS, UBEC2, HACL1, MSI2, and LOC100129585. In certain embodiments, RNA is associated with two or more, three or more, four or more, or all of the genes.

In certain embodiments, the probe or one or more probes are capable of hybridizing to an 8, 15, 30, 50, 100 or more base pair segment of the mRNA of a gene or RNA of a pseudogene. In certain embodiments, the probe or one or more probes are capable of hybridizing to the 5′ or 3′ terminal or interior segment. Typically the probes are conjugated to or capable of secondary detection by a fluorescent molecule. In certain embodiments, multiple fluorescent moieties provided barcoded probes or reporter probes hybridize directly to mRNA or RNA molecule in solution. The reporter probe allow for a light signal to provide information on the probe sequence after formation of a hybridization complex. This may be used in combination with a second probe or capture probe that contains a ligand which specifically binds a receptor immobilized to a solid surface. In certain embodiments, measuring is mixing a sample with a probe complementary to a segment of RNA or mRNA and measuring the binding of the probe to the RNA or mRNA.

In certain embodiments, GLS associated RNA is mRNA according to NCBI Reference Sequence: NM_014905.4 (Homo sapiens glutaminase (GLS), transcript variant 1, mRNA) or NCBI Reference Sequence: NM_001256310.1 (Homo sapiens glutaminase (GLS), transcript variant 2, mRNA).

In certain embodiments, UBEC2 (ubiquitin-conjugating enzyme E2C) associated RNA is mRNA according to NCBI Reference Sequence: NM_007019.3 (Homo sapiens ubiquitin-conjugating enzyme E2C (UBE2C), transcript variant 1, mRNA), NCBI Reference Sequence: NM_181799.2 (Homo sapiens ubiquitin-conjugating enzyme E2C (UBE2C), transcript variant 2, mRNA), NCBI Reference Sequence: NM_181800.2 (Homo sapiens ubiquitin-conjugating enzyme E2C (UBE2C), transcript variant 3, mRNA), NCBI Reference Sequence: NM_181801.3 (Homo sapiens ubiquitin-conjugating enzyme E2C (UBE2C), transcript variant 4, mRNA) NCBI Reference Sequence: NM_001281741.1 (Homo sapiens ubiquitin-conjugating enzyme E2C (UBE2C), transcript variant 7, mRNA), NCBI Reference Sequence: NM_001281742.1 (Homo sapiens ubiquitin-conjugating enzyme E2C (UBE2C), transcript variant 8, mRNA).

In certain embodiments, HACL1 (2-hydroxyacyl-CoA lyase 1) associated RNA is mRNA according to NCBI Reference Sequence: NM_012260.3 (Homo sapiens 2-hydroxyacyl-CoA lyase 1 (HACL1), transcript variant 1, mRNA), NCBI Reference Sequence: NM_001284413.1 (Homo sapiens 2-hydroxyacyl-CoA lyase 1 (HACL1), transcript variant 2, mRNA), NCBI Reference Sequence: NM_001284415.1 (Homo sapiens 2-hydroxyacyl-CoA lyase 1 (HACL1), transcript variant 3, mRNA), NCBI Reference Sequence: NM_001284416.1 (Homo sapiens 2-hydroxyacyl-CoA lyase 1 (HACL1), transcript variant 4, mRNA).

In certain embodiments, MSI2 (musashi RNA-binding protein 2) associated RNA is mRNA according to NCBI Reference Sequence: NM_138962.2 (Homo sapiens musashi RNA-binding protein 2 (MSI2), transcript variant 1, mRNA), NCBI Reference Sequence: NM_170721.1 (Homo sapiens musashi RNA-binding protein 2 (MSI2), transcript variant 2, mRNA).

In certain embodiments, LOC100129585 associated RNA is RNA of SEQ ID NO: 1 CCACCTACACGAGGGCGCCCCCATCTTATGGTGGAAGCAGTCGCTATGATGATT ACAGCAGCTCACGTGACGGATATGGTGGAAGTCGAGACAGTTACTCAAGCAGT CGAAGTGATCTCTACTCAAGTGGTCGTGATCAGGTTGGCAGACAAGAAAGAGG GCTTCCCCCTTCTATGGAAAGGGGGTACCCTCCTCCACGTGATTCCTACAGCAG TTCAAGCCGTGGAACACCAAGAGGTGGTGGCCGTGGAGGAAGCCGATCTGATA GAGGGGGAGGCAGAAGCAGATACTAGAAACAAACAAAACTTTGTACCAAAATC CCAGTTCAAAGAAACAAAAAGTGGAAACTATTCTATCATAACTACCCAAGAAC TACTAAAAGGAAAAATTGTGTTACCTTTTTTAAAATTCCCTGTTAAGCTCCCCTC CATAATTTTTATGTTCTTGTGAGGAAAAAAAGTAAAACATGTTTAATTTTATTTG ACTTTTGCATTGCTTTTCAACAAGCAAATGTTAAATGTGTTAAGACTTATACTAG TGTTGTAACTTTCCAAGTAAAAGTATCCCTAAAGGCCACTTCCTATCTGATTTTT CCCAGTAAATGAGGCAGGCAATTCTAAGATCTTCCACAAAACATCTAGCCATCT AAAATGGAGAGATGAATCATTCTACCTACACAAACAAGCTAGCTATTAGAGGG TGGTTGGGATATGCTACTCATAAGATTTCAGGGTGTCTTCCAACTGAAATCTCA ATGTTCTTAGTATGAAAAACCTGAAATCGCATGCCTATTCACCCAGTAAACCCA AAAAAGCAAATGGATAATGCTGGCCATTCTGCCTTTCTGACATTTCCTTGGGAA TCTGCAAGAACCTCCCCTTTCCCCTCCCCCAATAAGACCATTTAAGTGTGTGCTA AACAACTAAGAATACTAAAT.

In certain embodiments, the poly(ADP) ribose polymerase enzyme inhibitor is veliparib, iniparib, talazoparib, olaparib, or rucaparib. In certain embodiments, the platinum based reagent is cisplatin. In certain embodiments, cancer is lung cancer, nonsmall-cell lung cancer, small-cell lung cancer. In certain embodiments, the cancer is selected from the group consisting of leukemia, melanoma, cervical, ovarian, colon, breast, gastric, lung, skin, ovarian, pancreatic, prostate, head, neck, and renal cancer. In certain embodiments, the therapy includes one or more additional anticancer agents such as, but not limited to, gefitinib, erlotinib, docetaxel, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin and mithramycin, vincristine, vinblastine, vindesine, vinorelbine taxol, taxotere, etoposide, teniposide, amsacrine, topotecan, camptothecin bortezomib anegrilide, tamoxifen, toremifene, raloxifene, droloxifene, iodoxyfene fulvestrant, bicalutamide, flutamide, nilutamide, cyproterone, goserelin, leuprorelin, buserelin, megestrol anastrozole, letrozole, vorazole, exemestane, finasteride, marimastat, trastuzumab, cetuximab, dasatinib, imatinib, bevacizumab, combretastatin, thalidomide, and/or lenalidomide or combinations thereof.

In certain embodiments, the disclosure relates to methods of diagnosing a subject as a candidate for treatment with a poly (ADP) ribose polymerase enzyme inhibitor and a platinum based reagent comprising, measuring a quantity of RNA isolated from a cancer cell from the subject wherein the measurement indicates an increased quantity of the RNA compared to a normal sample, wherein the RNA is associated with one or more of the following genes GLS, UBEC2, HACL1, MSI2, and LOC100129585 and correlating the increased quantity to a diagnoses that the subject is candidate for treatment with a poly (ADP) ribose polymerase enzyme inhibitor and a platinum based reagent.

In certain embodiments, the disclosure relates to methods of diagnosing a subject as not a candidate for treatment with a poly (ADP) ribose polymerase enzyme inhibitor and a platinum based reagent comprising, measuring a quantity of RNA isolated from a cancer cell from the subject wherein the measurement indicates an increased quantity of the RNA compared to a normal sample, wherein the RNA is associated with one or more of the following genes/pseudogenes CENPE, CRYGS, FAM83D, FLJ44342, GNA12, LOC88523, LRDD, N4BP2L2, SLC35A3, SPC25 and correlating the increased quantity to a diagnoses that the subject is not candidate for treatment with a poly (ADP) ribose polymerase enzyme inhibitor and a platinum based reagent. In certain embodiments, RNA is associated with two or more, three or more, four or more, five or more, six or more, seven or more, eight or more, nine or more, ten or more, or all of the genes.

In certain embodiments, CENPE (centromere protein E) associated RNA is mRNA according to NCBI Reference Sequence: NM_001813.2 (Homo sapiens centromere protein E, 312 kDa (CENPE), transcript variant 1, mRNA), NCBI Reference Sequence: NM_001286734.1 (Homo sapiens centromere protein E, 312 kDa (CENPE), transcript variant 2, mRNA).

In certain embodiments, CRYGS (crystallin, gamma S) associated RNA is mRNA according to NCBI Reference Sequence: NM_017541.2 (Homo sapiens crystallin, gamma S (CRYGS), mRNA).

In certain embodiments, FAM83D (family with sequence similarity 83, member D) associated RNA is mRNA according to NCBI Reference Sequence: NM_030919.2 (Homo sapiens family with sequence similarity 83, member D (FAM83D), mRNA).

In certain embodiments, GNA12 (guanine nucleotide binding protein (G protein) alpha 12) associated RNA is mRNA according to NCBI Reference Sequence: NM_007353.2 (Homo sapiens guanine nucleotide binding protein (G protein) alpha 12 (GNA12), transcript variant 1, mRNA), NCBI Reference Sequence: NM_001282440.1 (transcript variant 2, mRNA), NCBI Reference Sequence: NM_001282441.1 (transcript variant 3, mRNA), NCBI Reference Sequence: NM_001293092.1 (transcript variant 4, mRNA).

In certain embodiments, LRDD (leucine rich repeat and death domain containing protein) is also known as PIDD1 (p53-induced death domain protein 1) associated RNA is mRNA according to NCBI Reference Sequence: NM_145886.3, transcript variant 1.

In certain embodiments, N4BP2L2 (NEDD4 binding protein 2-like 2) associated RNA is mRNA according to NCBI Reference Sequence: NM_033111.4 (transcript variant 1, mRNA), NCBI Reference Sequence: NM_014887.2 (transcript variant 2, mRNA), NCBI Reference Sequence: NM_001278432.1 (transcript variant 3, mRNA).

In certain embodiments, SLC35A3 [solute carrier family 35 (UDP-N-acetylglucosamine (UDP-GlcNAc) transporter), member A3] associated RNA is mRNA according to NCBI Reference Sequence: NM_012243.2 (transcript variant 1, mRNA), NCBI Reference Sequence: NM_001271684.1 (transcript variant 2, mRNA), NCBI Reference Sequence: NM_001271685.1 (transcript variant 3, mRNA).

In certain embodiments, SPC25 (SPC25, NDC80 kinetochore complex component) associated RNA is mRNA according to NCBI Reference Sequence: NM_020675.3 (Homo sapiens SPC25, NDC80 kinetochore complex component (SPC25), mRNA).

In certain embodiments, FLJ44342 associated RNA is GAGATGGTGCCCTTGATTAGAAGTGTCTGGAGGGGGATAAATGGAGGGGATAA GATTCAGTTGGTTTTGGAAAATGTTAAAGTCTTAAAATAATGCGTCCATCTGAA GAATTTTTTCTAAAACCAGAGTTTATAAAAATATCACTGATACAGCCTGCCCCC TCATTTCCCTGCCACAGGAGATGTCTTGGACTAGAGACACTTGTTTAATAATAG CTTGTCTCTGATATTCCCAGTAGCTTCCCTCTGTGTGAGGAAAGGATAGAAATG TTCAGGACATCATCATACAGGCTCCTCATCTACAAAGTTCCAGTAGCAGTGACG CCTACACGGAAGACTTGGAACTGCAAACAGGCTGGGGTCACCTCAGTGACATC TGACGCTGTCCAACCAGAAGTTCGATTTTTGTTCTGGGGGTGAAGGAGGAAACA GACTGTACTAAAGGACTAAAATAA (SEQ ID NO: 2).

In certain embodiments, LOC88523 associated RNA is GTGTGACTGAAGAAATATCAAATGTTTCCTAGTAAGACAGCAACTCA (SEQ ID NO: 3); ACTCTAGGATGGAAGAAGGTGTCTGACCGTAAATTACACCTGCAGT (SEQ ID NO: 4); AACCAGCAGACTAATGGGGATGAGGTTCTGGTACAAGATGATGAACACCAGTA TGTCAGACAATGACTTGGGAGCTGGAATCAAGGACATGACCAAGAGCAGCAAG AACAAAAGGGAGACTGACACATTGATCACTTTCTCAACCTTTGATCTCTTGAGA (SEQ ID NO: 5); ATGGTTCACATTTGAGTAAAGACAGGGGAGTTTGTTTTCAGAATGACATACTAG TCTGCAGGATGAATTTCATAACTGACATTGCACCTTGGACTGCAACTAGGACTT TCACTGGAATCA (SEQ ID NO: 6); GAAAGAGTTTTGAAGAAAACTGGGCATAGGCTCAGCAAAACCAAACAGAAGA GGAACAGAAAAAGAAACAAAAAGCAGAACAGTCAGAATAGAATCATGGAGGA AAACTCATTAGAATTCTTAAGTGATCTTACACCGGGAGATCAGGACCCATCTCA GAGTGAAGAGGAAGACATTGAAAAGACCAGAAGAGAATCAGAATATCCCTTCA TTGATGGTCTACAAAATGAAGTCGGAGATTTTGTGACTGGATATAAAGAAAAA AGATGGAAAAATAAAGATCCTAAAGACAGTTTCCAAAACGTTATGTCTATAGTT GAATTAGACAACACACCAAAGAATTACCTCTCTAAGGAAGGTGATAACTTGTTT GTAAGTTTGTTACTGAGGCCAAATGAAATCTCCGTTACTTGTCCAATACTGACT CAAAACCTTTCCTGTGTAACAACTGATGACTGCTCTGGCATGAAGGTAGAAAAG CATATTAGAAATAGGCATACCATAGCATTAGACACCCAGGACCTTTCTGCGGAA ACTTCATGCTTATTTATGAAGAAGAGAGAAATAGTAGATAAAAATCTCTCACAT GAACCCATTCTGTGCCATCAACATGGAATCAGAATGTCAGATAAAGTTTTAAGA GAGGAACAAGTGTATACAACTAAAATCAATCACTGGGCTTTTTTCACAACCAAT TTATCTGATGAAGATTTACAGCTGGGCTCTGACAGACAGCCCTATTTTGGTAGC TGGCCTGCAGGACCTCATAAGTTTATATGTGAACAGAGACCAAAGAAAGATAG AGCATGTAAGTTGGCTGGTCCTGACAGCAGGGGGCAATGGATTCAAATGATCTT CACTTCGGTGGCAGCATCAGAACCAGGAAACAATCCAGAAATATTGACAGACA AACTACTGATAGGAAATGAAGATTTTTCACCTCCACCTGAAACTATGGATTCAT TCATAGAAACAAACCTCTTCAGAAGCTGCTTACCTCAACCGGATATACCAAAGA ATGCCTTAGAATCAACAAAAAATAAGAAAAGGAGGAAGAAAAGGATTTTCAAT TTGGTACCAAATTTTGACTTATTAGGACAGAGTCGTATCGGTGTAAAAGAAAGG GAGAAATGTGACCTGTTAACAAAAAACCATGGACTAAAAATTACTTTGGGAGA AGAAAAAGATAGAATTTCAGAAAGGAACAGTGAAGAGGAGAATAAACAAAAA CTTATGACCTTTGATCATCATCCATTGTGGTTTTACCTTGATATTATCAAAGCTA CCCCTTTAAATATTGATGGACAGCGTTATTCTCATTGCCTGTCATTTAACAGACT AAGGTGCTCTGCATCTTTATACAAAAATTATATTCCTTCTTTTGTGCTACATAAT TTATCTAGTATTTGGAAGCCATCTTTTACAAACAAGAAACTGTTTTTGACTTTCG AATCTCAGACAAGAGTAGGTAATAAACTAAATGATGCAGGGTTTATTTCTCCAG AAATTTTACATAGTCATCCTGATACTTCGTGCTCTTTGGGAGTCACTTCTGATTT TCACTTTTTAAATGAAAGGTTTGATAGAAAGCTGAAAAGATGGGAAGAACCTA AGGAATTACCAGCTGAGGACAGCCAAGACTTAACAAGCACTGACTACCGTTCC CTTGAGCTACCATTATCACAAGGGTTTGCCTTTCAATTAGTAAAGCTTTTTGGAT CTCCAGGCGTTCCAATG (SEQ ID NO: 7); ATCCTTGTTGCCTGATGACTATGTGGTTCCCCTTGACTGGAAGACACTAAAGAT GATCTACTTGCAATGGAAGATGTCAGTGGA (SEQ ID NO: 8); AAAGACAGAAGAAGATTGGTTGAAAAATGAAAATTCCTTGAAC (SEQ ID NO: 9) TGAGTTCTGCTGTCTTCATGGTACTGCTGAAGATCATGATCACGGAGAAAAGTC AGAGTGCTCAGTGCCAACCCAAGGGATTCTTTCCAGAGACGTACCCGTTGGATA CCAAAATTAGTTTGGATAATCTGTTCAACCATTCTTGATAAGTTATCTGAATAAT AAAAAAACTCAACAGAGGAGGTAACAATTTGAACATTTTATTGTCTAATTTGAA GATGTATGCCATACTTTGTTTGATAGAAGAAAGTAAGGCACAGAAAACTTGAGT ACCTTATTTTTAAAACTGCATTAGGATTAAAAGGTTAGCCCCTATATCCAAGTA TTGGTCTGAGATCCCATTTCTAGAATTCTGAAATCCAAAAAGCTCTGAAAATCA ATAGTTATTTTTCCAAATGTATTCATTGTGGTAAAATACACCAATATAAAATTTA CCATCTTAACCACTTTTAAGTGTGTTATAAATACATTCATGCTACCATCACTAGC ATCCATCTCTGGAACTCTTTTCATCTTGCAAAACTGCAATTCTATACCCATTAAA CAATGACCCA (SEQ ID NO: 10)

### Measuring RNA Expression

Measuring RNA expression can be done by any variety of methods known in the art such as but not limited to using polymerase chain reaction (PCR), northern hybridization (or northern blotting), expressed sequence tag (EST), serial analysis of gene expression (SAGE), representational difference analysis (RDA), differential display, suppression subtractive hybridization (SSH), nucleic acid immobilized microarrays, RNA-seq, or single-cell RNA detection methods.

An RNA sample may be purified prior to detection. For example, mRNA typically contains polyadenine tail present at the 3′ end. One can use poly-T oligonucleotides that hybridize to the complementary poly-A tails that are immobilized on solid supports to purify mRNA. Sample RNA may also be separated by gel electrophoresis. The separated RNA may be transferred to a membrane and exposed to labeled probes. Hybridization of complementary probes allows visualization of target RNA sequences.

In certain embodiments, the disclosure contemplates measuring RNA by PCR or direct hybridization of a probe that comprises a detectable moiety, e.g., optical reporter. Other contemplated methods include quantitative PCR wherein the amplified nucleic acids are detected as the reaction progresses in “real time.” For example, non-specific fluorescent dyes can intercalate within cDNA that is the result of PCR amplification, or sequence-specific probes consisting of oligonucleotides may be labelled with a fluorescent reporter which permits detection after hybridization of the probe with its complementary sequence. Fluorescent probes can be used in multiplex assays for detection of several genes in the same reaction based on specific probes with different-colored labels. In certain embodiments, this method utilizes a probe/primer with a fluorescent reporter at one end and a quencher of fluorescence at the opposite end of the probe/primer. The close proximity of the reporter to the quencher prevents detection of its fluorescence; breakdown of the probe by the 5′ to 3′ exonuclease activity of the Taq polymerase breaks the reporter-quencher proximity and thus allows unquenched emission of fluorescence, which can be detected after excitation with a laser. An increase in the product targeted by the reporter probe at each PCR cycle therefore causes a proportional increase in fluorescence due to the breakdown of the probe and release of the reporter.

Quantification using microarrays are based on the precise immobilization oligonucleotides probes at high density on surface-modified solid supports. Probes may hybridize with target nucleic acids that have been labeled during a reverse transcription (RT) procedure. Hybridized targets reflect the amount of RNA isolated from a sample. Fluorescence emitted by each spot is proportional to the amount of RNA in the sample.

Single-cell analysis of RNA may be accomplished by in situ hybridization (ISH), whereby labeled linear oligonucleotide (ODN) probes are used to label intracellular RNA in cells that are fixed and permeabilized. Multiple probes may be used to target the same RNA. The absolute number of RNA per cell can be quantified. Other contemplated methods include the use of tagged linear probes, linear FRET probe pairs, molecular beacons, dual FRET molecular beacon pairs, quenched autoligation probe pairs, and fluorescent protein based probes. See Bao et al., Fluorescent Probes for Live-Cell RNA Detection, Annu Rev Biomed Eng, 2009, 11:25-47.

In certain embodiments, the disclosure contemplates quantification using serial analysis of gene expression (SAGE), LongSAGE, RL-SAGE, and SuperSAGE. Velculescu et al., Science, 1995, 270: 484-487 and Matsumura et al., Nat Methods, 2006, 3(6):469-74.

In certain embodiments, the disclosure contemplates quantification using RNA-seq techniques. RNA-Seq based methods utilize RNA that converted to a library of shorter random cDNA fragments with adaptors attached to one or both ends. Each molecule is sequenced by a variety of methods that manipulate the properties of the adaptors providing shortened overlapping sequences. These are reassembled typically by comparisons to known DNA and RNA sequences. See, Wang et al., RNA-Seq: a revolutionary tool for transcriptomics, Nature Reviews Genetics, 2009, 10, 57-63. Islam et al., Quantitative single-cell RNA-seq with unique molecular identifiers, Nat Methods, 2014, 11(2):163-6.

### Kits

In certain embodiments, the disclosure contemplates kits comprising probes and primer pairs that hybridized to the RNA sequences or nucleic acid binding proteins. Typically, the probes or primer pairs are more than 8, 9, 10, 11, 12, 13, 14, or 15 nucleotides long. Typical probes include linear, double stranded, or hairpin oligonucleotides with a reporter, e.g., fluorescent dye. In certain embodiments, pairs of fluorescence resonance energy transfer (FRET) probes are contemplated. Typically the first probe contains a fluorescent dye, a second contains a quencher. When not bound to the target nucleic acid the first probe produces light. The first and second probe is configured to bind in close proximity to each other such that the quencher on the second probe quenches the light produced from the first probe. The first and the second probes may be in the form of a single oligonucleotide hairpin sometimes referred to as dual FRET molecular beacon. The first and the second probes may be in the form of autoligation FRET probes, e.g., one labeled with a FRET acceptor (e.g., Cy5) and a nucleophile, and the second labeled with a FRET donor (e.g., FAM) and an electrophilic dabsyl quencher. Upon binding of the two probes to adjacent sites on the same RNA, the quencher is displaced and a ligation brings the donor and acceptor fluorophores together, resulting in FRET signal because the nucleophilic group displaces the dabsyl group via nucleophilic substitution reaction. RNA-binding proteins (RBPs) tagged with optical reporters such as green fluorescent protein (GFP) can be used to bind probes using fluorescent proteins as reporters. The probe contains a segment that binds the target nucleic acid and a second reporter segment that forms a stem loop recognized by the RNA-binding protein conjugated to the optical reporter.

In some embodiments, the probe includes at least one fluorophore. In other embodiments, the probe includes at least two fluorophores. In such embodiments, the two or more fluorophores can be in close proximity, and in some embodiments excitation of one fluorophore can lead to excitation of a second or further fluorophores. Examples of fluorophores include but are not limited to Cy2, Cy3, Cy3B, Cy3.5, Cy5, Cy5.5, Cy7, FAM, 6-FAM, Fluorescein, JOE, TET, HEX, TRITC, Texas Red, X-Rhodamine, Lissamine Rhodamine B, Allophycocyanin (APC), BODIPY-FL, FluorX, TruRed, PerCP, Red 613, R-Phycoerythrin (PE), NBD, Lucifer Yellow, Pacific Orange, Pacific Blue, Cascade Blue, Methoxycoumarin, Aminocoumarin, and Hydroxycoumarin.

In some embodiments, the probe includes at least one quencher. In some embodiments, the quencher is a non-fluorescent quencher including but not limited to a Black Hole Quencher (BHQ), Eclipse Dark Quencher (DQ), IOWA Black (IWB), DABCYL, and TAMRA.

Contemplated probes or primers may be configured as hairpin loops such that the segment that hybridizes to the nucleic acid sequences are inside the loop. For example, in certain embodiments, the kit comprises primer pairs and probes that bind RNA associated with GLS, UBEC2, HACL1, MSI2, and LOC100129585.

In certain embodiments, the probes are single stranded oligonucleotides that have terminal segments that self-hybridize having a fluorescent dye and a quencher on opposing terminal segments, often referred to as dual-labeled oligonucleotide hairpin probes or molecular beacons. A typical molecular beacon probe is a hairpin loop between 18 and 40 nucleotides or longer. The middle 8-20 nucleotides are complementary to the target nucleic acid and do not base pair with one another, while the nucleotides at each terminus are complementary to each other rather than to the target nucleic acid. A typical loop has a 8-30 base pair region that is complementary to the target nucleic acid. The stem is formed by the internal hybridization both termini of the loop, of two short (5 to 7 nucleotide residues) oligonucleotides that are complementary to each other. Near the first 5′ or 3′ end, a fluorescent dye is covalently attached. Near the other end 3′ or 5′ end respectively is a quencher (e.g., non-fluorescent). When the beacon is in closed loop shape, the quencher resides in proximity to the fluorophore, which results in quenching the fluorescent emission of the latter.

If the nucleic acid to be detected is complementary to the strand in the loop, the event of hybridization occurs. The duplex formed between the nucleic acid and the loop is more stable than that of the stem because the former duplex involves more base pairs. This causes the separation of the stem and hence of the fluorophore and the quencher. Once the fluorophore is separated from the quencher, light illumination of the hybridized complex results in a fluorescent emission. The presence of the emission reports that the event of hybridization has occurred and hence the target nucleic acid is present in the test sample.

Primers and probes may be arranges such that they may be detected through secondary detection. The terminal ends of primers may contain adaptors, e.g., additional sequences inserted that cause PCR amplification to include tags or unique hybridization sites on the terminal ends of the amplified nucleic acid. Thus, the amplified nucleic acid can be further detected through binding of complementary labeled nucleic acids, e.g., molecular beacons configured to hybridize with the terminal hybridization sites as described above.

### EXPERIMENTAL

### Veliparib Pharmacokinetic and Platinum Adducts

Tumor-bearing animals were treated with a single dose of vehicle, veliparib (5 mg/kg or 25 mg/kg), cisplatin (2.5 mg/kg or 5 mg/kg), and combinations. Treated animals were sacrificed either at 1 or 24 h posttreatment by cervical dislocation. Plasma and tumor samples were collected and immediately stored in liquid phase nitrogen or at 70° C. until ready for analysis. Tissues were homogenized in approximately 1 mL of PBS. Veliparib concentrations in plasma and tissue homogenates were quantitated by LC-MS. Concentrations of total platinum in plasma and tissue homogenate were quantitated by atomic absorption spectrophotometry (AAS).

### Veliparib Displayed Limited Single-Agent Activity In Vitro but Potentiated the Cytotoxicity of Cisplatin, Carboplatin, Etoposide, and Ionizing Radiation

Short-term MTS cytotoxicity assay was performed to characterize veliparib activity in a panel of 9 SCLC cell lines. Veliparib induced limited growth inhibition over a wide concentration range (0-128 μmol/L) in the panel of SCLC cell lines tested (FIG. 1). There was modest activity in several cell lines (H187, H146, DMS153) especially at concentrations ≧20 μmol/L. Veliparib at a concentration of 50 μmol/L but not at 5 μmol/L potentiated the activity of cisplatin, carboplatin, and etoposide leading to a ≧50% reduction in the IC₅₀ concentration of the cytotoxic drugs in five of nine cell lines (FIG. 1). There was a positive correlation of the magnitude of potentiation by veliparib and the sensitivity of the cell line to the cytotoxic agent, especially with cisplatin i.e., the lower the single agent IC₅₀ the greater the degree of potentiation in the specific cell lines: CC=0.67, 0.22, and 0.24 for cisplatin, carboplatin, and etoposide, respectively. Similar potentiation of radiation-induced cytotoxicity was noted when veliparib (5 μmol/L) was combined with two different doses (2 and 4 Gy) of ionizing radiation in two representative cell lines (DMS153 and H146).

### The Combination of Veliparib and Cisplatin Achieved Greater Tumor Growth Inhibition in SCLC Xenografts

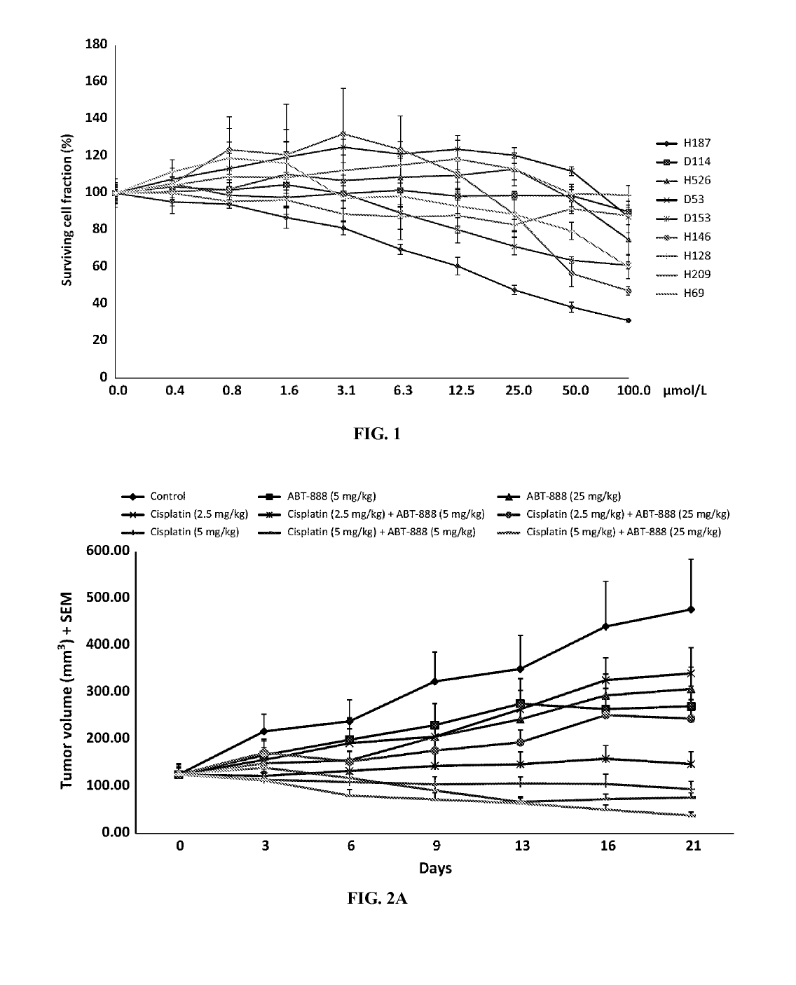

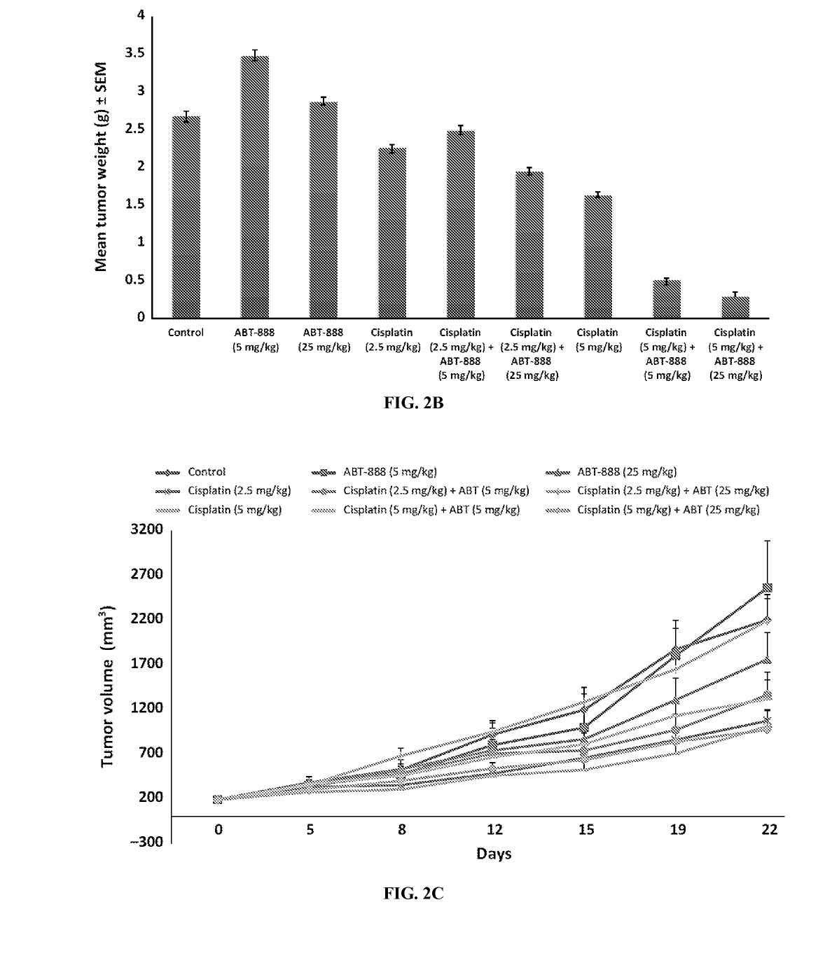

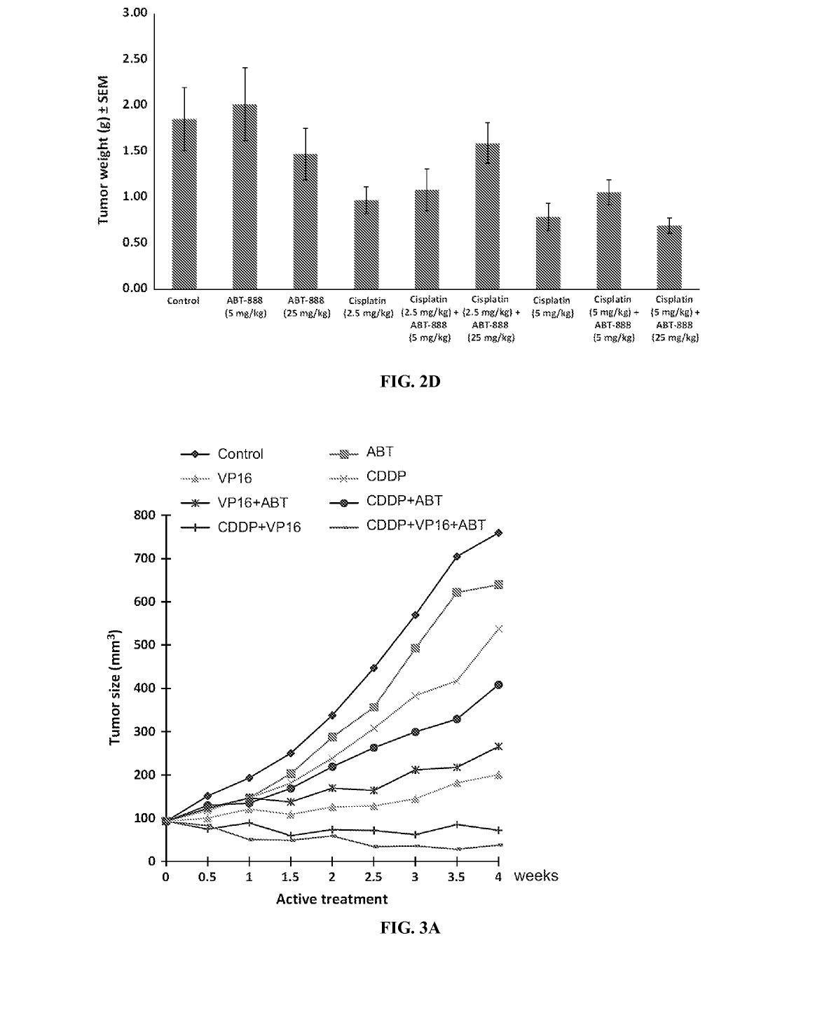

The potentiating effect of veliparib on cisplatin was tested in vivo. Two SCLC cell lines with a threefold difference in sensitivity to cisplatin based on the IC₅₀ concentration H146 (5.2 μmol/L) and H128 (14.5 μmol/L) were used from the in vitro assay for this in vivo experiments. There was greater tumor growth inhibition with the veliparib and cisplatin combination than with cisplatin alone in H146 xenografts (FIGS. 2A and B; P=0.09) but not in the H128 xenograft (FIGS. 2C and D; P>0.1). The potentiating effect of veliparib when combined with cisplatin appeared dose dependent (FIG. 2B) but without additive toxicity as indicated by the measured weight of the animals.

The Veliparib, Etoposide, and Cisplatin Combination was More Potent than Cisplatin and Etoposide Alone in Preventing Tumor Regrowth Post-Treatment

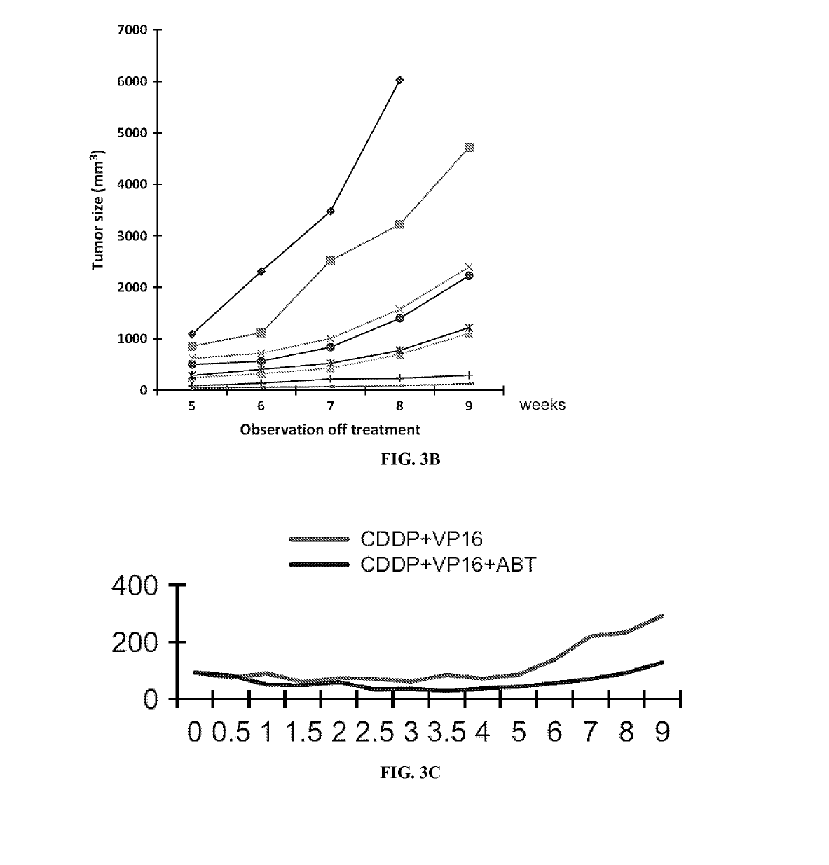

Patients are typically treated with the combination of platinum and etoposide and not with single-agent platinum. The addition of veliparib to the platinum doublet (cisplatin and etoposide) was studied in vivo. The triplet combination of veliparib, cisplatin, and etoposide was more potent than the doublet (P=0.07) and induced objective tumor regression while the doublet only reduced tumor growth. Moreover, the triplet regimen significantly delayed tumor regrowth over the cisplatin and etoposide doublet when treated animals were observed off treatment for up to 4 weeks (P=0.02; FIG. 3A-C).

Expression Profiling on Illumina HT2 and nCounter NanoString Platforms and Bioinformatics

Each cell line was treated with vehicle, veliparib (5 and 50 μmol/L), cisplatin (determined IC50 concentration for each cell line), ionizing radiation (2 Gy) or cisplatin plus veliparib combination for 24 h. Total RNA was isolated from frozen specimens using RNeasy (Qiagen, Valencia, Calif., USA) according to the manufacturer's instructions. Total RNA sample quality and concentrations were determined using NanoDrop and Agilent 2100 Bioanalyzer. Each sample was prepared for Illumina Human HT-12 v4 Expression BeadChips (Illumina, San Diego, Calif., USA) according to the manufacturer's protocol. The HT-12 platform contains over 47,000 probes that cover characterized genes, gene candidates, and splice variants. BeadChips were scanned on the Illumina HiScan instrument to determine probe fluorescence intensity. Raw probe intensities for all treatment conditions were normalized by the quantile normalization algorithm using GenomeStudio software from Illumina and log-2 transformed expression obtained for analyses. An unsupervised cluster analyses was done to examine the relatedness, genome-wide, among the cell lines and treatment conditions for identifying any outlying samples. Results were compared between treatment conditions to define commonly altered genes in both PARP inhibitor sensitive and insensitive cell lines.

Both a semiparametric analysis of variance (ANOVA) and a nonparametric, variance approach were implemented to obtain a robust (to analytical assumptions) gene list that was supplemented with additional genes of research interest. For the ANOVA, a mean comparison of expression was done, where feasible, to test expression differences within and among treated cell lines to controls. Results from this approach are based on an unadjusted P<0.01 and a fold change of at least 1.5. Separate variance analyses were done in which empirical distributions of expression variance within each gene was performed in order to identify specific genes whose variance was among the top and bottom percentile relative to all genes (high and low variability, respectively). Genes with high expression variability among designated “sensitive” cell lines within treatment were considered as susceptible to treatment. Likewise, genes with low expression variability were considered nonresponsive to treatment. Several comparisons of results were made within and between treatments with respect to expression variability and testing for mean differences in expression based on the ANOVA results. These data were deposited in NCBI Gene Expression Omnibus as series GEO accession GSE55830.

nCounter Nanostring Gene Expression

The expression of 129 genes including 31 DNA repair genes and 38 high or low variability genes from the Illumina HT-12 expression data analysis was determined using NanoString nCounter Gene Expression platform (NanoString Technologies, Seattle Wash.) at the University of Miami Oncogenomics Core facility. The design and synthesis of probe sets for the 129 selected genes were performed at NanoString Technologies. In addition to the data from the nine cell lines, patient samples from 81 pulmonary neuroendocrine tumors (17 carcinoid, 11 large cell carcinoma, 40 small cell carcinoma, 13 neuroendocrine cancer) were included in the expression assay. Data preprocessing involved the following: an initial correction for batch assignment using the sum of the positive controls, subtraction of background signal defined by the mean expression of the negative controls, log-2 transformed, zero-centered, and quantile normalized. Samples containing greater than 75% zero expression values were removed prior to quantile normalization.

### Gene Expression Profiling Characterized SCLC Cell Lines Sensitive to PARP Inhibition

The gene expression profile of the sensitive and the less sensitive cell lines were compared in their native state and under various treatment conditions. Unsupervised cluster analysis of Illumina HT-12 data comparing the baseline gene expression profile of untreated SCLC cell lines showed tight clustering of 5 cell lines (H146, H187, H209, H526, and DMS114), which were mostly the same cell lines that displayed increased sensitivity to cisplatin and to PARP inhibition (arbitrarily defined as at least 50% reduction in the IC₅₀ concentration of cisplatin when combined with veliparib). Unsupervised analysis of the gene expression profiles of the cell lines under different treatment conditions showed cells clustering by cell of origin rather than by treatment. A hierarchical supervised analysis of the gene expression profile of the two clusters of cells (PARP inhibitor sensitive vs. PARP insensitive) before and after exposure to the optimal concentrations required for cytotoxicity i.e., cisplatin (IC₅₀) and veliparib concentrations (50 μmol/L), revealed a panel of 24 genes and pseudo genes (27 probe sets) with differential expression between the two cell clusters. Five of these genes were restricted to the sensitive cell lines (GLS, UBEC2, HACL1, MSI2, and LOC100129585), 9 were restricted to the insensitive cell lines (CENPE, CRYGS, FAM83D, FLJ44342, GNA12, LOC88523, LRDD, N4BP2L2, SLC35A3, SPC25) and the remaining genes were common to both groups (AURKA, CENPA, DLGAP5, HMMR, KIF20B, LOC100129585, LOC100131735, RBMX, SFRS3. It is contemplated that this panel of genes either alone or in combination may identify the cell population likely to be sensitive to cisplatin and/or the combination of a PARP inhibitor and DNA damaging agents.

What we claim:

1. A method of treating cancer comprising administering an effective amount of a poly (ADP) ribose polymerase enzyme inhibitor and a platinum based reagent to the subject in need thereof, wherein the subject is in need thereof because measuring a quantity of RNA isolated from a cancer cell from the subject indicates an increased quantity of the RNA compared to a normal sample, wherein the RNA is associated with one or more of the following genes GLS, UBEC2, HACL1, MSI2, and LOC100129585.

2. The method of claim 1, wherein the poly (ADP) ribose polymerase enzyme inhibitor is veliparib.

3. The method of claim 1, wherein the platinum based reagent is cisplatin.

4. The method of claim 1, wherein the cancer is lung cancer.

5. The method of claim 1, wherein the RNA is associated with two or more, three or more, four or more, or all of the genes.

6. A method of diagnosing a subject as a candidate for treatment with a poly (ADP) ribose polymerase enzyme inhibitor and a platinum based reagent comprising,
measuring a quantity of RNA isolated from a cancer cell from the subject wherein the measurement indicates an increased quantity of the RNA compared to a normal sample, wherein the RNA is associated with one or more of the following genes GLS, UBEC2, HACL1, MSI2, and LOC100129585 and
correlating the increased quantity to a diagnoses that the subject is candidate for treatment with a poly (ADP) ribose polymerase enzyme inhibitor and a platinum based reagent.

7. The method of claim 6, wherein the poly (ADP) ribose polymerase enzyme inhibitor is veliparib.

8. The method of claim 6, wherein the platinum based reagent is cisplatin.

9. The method of claim 6, wherein the cancer is lung cancer.

10. The method of claim 6, wherein the RNA is associated with two or more, three or more, four or more, or all of the genes.

11. A method of diagnosing a subject as not a candidate for treatment with a poly (ADP) ribose polymerase enzyme inhibitor and a platinum based reagent comprising,
measuring a quantity of RNA isolated from a cancer cell from the subject wherein the measurement indicates an increased quantity of the RNA compared to a normal sample, wherein the RNA is associated with one or more of the following genes CENPE, CRYGS, FAM83D, FLJ44342, GNA12, LOC88523, LRDD, N4BP2L2, SLC35A3, SPC25 and
correlating the increased quantity to a diagnoses that the subject is not candidate for treatment with a poly (ADP) ribose polymerase enzyme inhibitor and a platinum based reagent.

12. The method of claim 11, wherein the poly (ADP) ribose polymerase enzyme inhibitor is veliparib.

13. The method of claim 11, wherein the platinum based reagent is cisplatin.

14. The method of claim 11, wherein the cancer is lung cancer.

15. The method of claim 11, wherein the RNA is associated with two or more, three or more, four or more, or all of the genes.
